(19) United States
(12) Patent Application Publication
(10) Pub. No.: US 2023/0351073 A1
(43) Pub. Date: Nov. 2, 2023

(54) SYSTEMS AND METHODS FOR OCULAR FINITE ELEMENT MODELING AND MACHINE LEARNING

(71) Applicant: Ace Vision Group, Inc., Newark, CA (US)

(72) Inventors: AnnMarie Hipsley, Newwark, CA (US); Laurent Sabatier, Cocoa Beach, FL (US); James Emmett O'Flanagan, Newark, CA (US)

(21) Appl. No.: 18/305,125

(22) Filed: Apr. 21, 2023

Related U.S. Application Data
(60) Provisional application No. 63/334,107, filed on Apr. 23, 2022.

(51) Int. Cl. G06F 30/23

(52) U.S. Cl. CPC G06F 30/23

(57) ABSTRACT

Hardware and software system solutions provide real-time, interactive predictive simulations of the eye (e.g., human eye or animal eye). The predictive simulations of an individual's eye can be created using a Finite Element Model (FEM) of ocular structures involved in optical biomechanics, including ocular accommodation.

### CROSS REFERENCE TO RELATED APPLICATIONS

This application claims the benefit of priority to U.S. Provisional Application No. 63/334,107 filed Apr. 23, 2022 entitled “Systems and Methods for Ocular Finite Element Modeling and Machine Learning,” the entire contents of which is incorporated by reference herein in its entirety for all purposes.

### TECHNICAL FIELD

The subject matter described herein relates to ocular finite element modeling (FEM) and machine learning, and more specifically, a virtual ecosystem configured to provide real time, dynamic interactive virtual ocular image recreation in virtual reality of a living eye in a plurality of conditional states using several related predictive simulation algorithms. Interactive virtual ocular image is capable of representing individual specific mechanical, material, and physics properties and demonstrating and/or mimicking the behavior of the living eye (regardless of species) under a variety of conditions and imposed demands namely surgical intervention, therapeutic intervention, representative progression of disease processes. Further, this invention is capable of relaying numerical simulations and results of these various interventions including the reversal of interventions and or disease processes. Further, this invention is configured to apply numerical methods and or calculations on any data source(s) in order to predict a plurality of expected and or unexpected conditions and events in the living eye.

### BACKGROUND

The eye is a biomechanical structure that contains complex muscular, drainage, and fluid mechanisms responsible for visual function and ocular biotransport. The accommodative system is the primary moving system in the eye organ, facilitating many physiological and visual functions in the eye. The physiological role of the accommodation system is to move aqueous, vitreious, blood, nutrients, oxygen, carbon dioxide, large and small molecule drugs and other cells, around the eye organ.

In general, the loss of accommodative ability in presbyopes has many contributing lenticular, as well as extralenticular and physiological factors that are affected by increasing age. Advanced Glycation End products (AGEs)=Accumulation of AGE's create detrimental chemical bonds that cause increased biomechanical stiffness in the connective tissues of the body including the eye.

Increasing ocular rigidity with age produces stress and strain on these ocular structures and can affect accommodative ability which can impact the eye in the form of decreased biomechanical dysfunction for physiological processes including visual accommodation, aqueous hydrodynamics, vitreous hydrodynamics and ocular pulsatile blood flow to name a few. Current procedures only manipulate optics through some artificial means such as by refractive laser surgery, adaptive optics, or corneal or intraocular implants which exchange power in one optic of the eye and ignore the other optic and the importance of preserving the physiological functions of the accommodative mechanism.

Additionally, current implanting devices in the sclera obtain the mechanical effect upon accommodation. They do not take into account effects of pores (e.g., micropores) or creating a matrix array of pores in 3D tissue. As such, current procedures and devices fail to restore normal ocular physiological functions.

Accordingly, there is a need for improved systems and methods for restoring normal ocular physiological functions.

### SUMMARY

In general, hardware and software system solutions are presented herein that provide real-time, interactive predictive simulations of the eye (e.g., human eye or animal eye). As described in more detail below, the predictive simulations of an individual's eye can be created using a Finite Element Model (FEM) of ocular structures involved in optical biomechanics, including ocular accommodation.

In one aspect, there is disclosed a system for recreation , manipulation and intervention of an individual's eye in virtual reality comprising: a 3D modeling component comprising of at least one data processor; an imaging system, a biothermal system, an interactive user interface; an inputs program to receive and manipulate physical, biometric, biomechanical, material and mechanical information and data; and at least one memory storing instructions, which when executed by the at least one data processor, result in operations comprising: generating a first specific simulation in which the Bruch's membrane Choroid is activated to determine the first contribution of the energy required for deformation of the lens; generating a second individual-specific simulation in which a origin section of the ciliary muscle fiber section of an eye of the individual is activated to determine a first contribution of the first ciliary muscle fiber section to a deformation of a lens of the eye; generating a second individual-specific simulation in which a second ciliary muscle fiber section of the eye of the individual is activated to determine a second contribution of the second ciliary muscle fiber section to the deformation of the lens of the eye; generating a third individual-specific simulation in which a third ciliary muscle fiber section of the eye of the individual is activated to determine a third contribution of the second ciliary muscle fiber section to the deformation of the lens of the eye; and determining, based at least on the first Bruch's Membrane Choroid Complex (BMCC) apparatus, the first contribution of the first ciliary muscle fiber section, the second contribution of the second ciliary muscle fiber section, the third contribution of the third ciliary muscle fiber section, one or more parameters of a treatment for the individual. All simulations are capable of being performed, in sequence, in isolation and/or in parallel with other parameter manipulations. The system can further generate a fluid structure interaction of the aqueous flow of the eye in which three outflow pathways are activated. The system includes one or more parameters of the Bruch's Choroid Apparatus, the first ciliary muscle fiber section and/or the second ciliary muscle fiber section and /or the third ciliary muscle fiber section of the eye of the individual. In an embodiment, the one or more micropores are generated into the scleral tissue of the eye over the Bruch's Choroid apparatus, the first ciliary muscle fiber section and/or the second ciliary muscle fiber section and/or third ciliary muscle fiber section of the eye of the individual by laser. The one or more parameters of the treatment can include a quantity of matrices including the one or more pores, a placement of the matrices, an overall shape of each matrix, an overall dimension of each matrix, a quantity of pores in each matrix, a distribution of pores in each matrix, and/or a depth of pores in each matrix. In an embodiment, the one or more treatment parameters for the individual are determined by applying a machine learning model, such as a regression model and algorithm or an age progression model and algorithm. The machine learning model can be trained based on various combinations of treatment parameters and treatment outcomes. The machine learning model is further trained based individual demographics data associated with the various combinations of treatment parameters and treatment outcomes. A user interface can be generated displaying one or more parameters of the treatment for the individual. The system can further comprise receiving, via the user interface, at least one user input associated with the one or more parameters of the treatment for the individual; and updating, based at least on the user input, the machine learning model.

The operations further comprise systems and method for provided for evaluating a biomechanical property of tissue, a simulated generator which incorporates various optical, biomechanical, physical characteristics of a given individual's eye into a simulated VR phantom of the living eye, and/or a parameter calculation component calculates a value for the biomechanical properties at a plural subset of the plurality of locations in the eye (Hoop stress map) wherein the virtual eye is interactive, and/or physics calculations of the eye. An evaluation toolbox component is configured to calculate at least one parameter associated with the various possible outcomes of a given treatment intervention in an individual's eye from the extracted plurality of features. A system output is configured to provide the calculated at least one parameter to one of a treatment system and a user. One or more optical, biometric, biomechanical, physical, and geometrical parameters are incorporated into the calculation prediction. The calculator toolbox of VESA is capable of reconciling equations that produce the most optimal solution(s) therapeutic or surgical intervention to the user.

In another aspect, there is disclosed a system for evaluating an eye of a patient, comprising: a modeling component configured to determine a representation of the whole eye from a three-dimensional structural image of the whole eye and at least one biomechanical property of the eye; a feature extractor configured to extract a plurality of features from the model of the whole eye; a user interface configured to accept input from a clinician defining an objective function as a function of at least one parameter for the eye after the therapeutic procedure; an condition or disease evaluation component configured to calculate at least one parameter associated with the risk of the disease in the eye from the extracted plurality of features and the objective function, the calculated at least one parameter including a variable in a therapeutic procedure, treatment intervention, and/or surgical process representing a surgical parameter that can be varied by a clinician in the therapeutic procedure; and a system output configured to provide the calculated at least one parameter to one of a treatment system and a user.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1A shows VESA, 3D-ID AI, MP Tool, and Regression Equation Database and their relationships to one another.

FIG. 1B illustrates and exemplary process flow chart.

FIG. 2A illustrate a VESA ecosystem having various hardware/software systems residing in the procedure room, the cloud, and/or database.

FIG. 2B illustrates the various technologies and tools that make up the backend services of the VESA ecosystem.

FIG. 2C illustrates a plurality of sub-systems of the VESA ecosystem that includes standalone diagnostic tools, clinical trial tools, lasers, imaging devices, AI engines, etc.

FIG. 2D illustrates two of the many disease application modules.

FIG. 3 illustrates VESA contributions at various stages of a key medical procedure.

FIG. 4 illustrates the structures, various measurable variables, and boundary conditions that can be used to generate the finite element model of the eye.

FIG. 5 illustrates exemplary zone definitions that can be used to generate the finite element model. The zone definitions can be applied to any part of the eye including a posterior region, anterior region or any other region.

FIG. 6 illustrates examples of each of the zone description or anatomical location with respect one or more areas of the eye.

FIG. 7 illustrates an example 3D model generated by VESA.

FIG. 8 illustrates a FEM created using more fibers and elements.

FIG. 9 illustrates an example mesh network of a 3D model of an eye.

FIGS. 10-12 illustrates example anatomy models used by VESA to generate the trabecular meshwork, or any other aspect of the eye including for example ciliary zonules, lens or any other eye anatomy.

FIG. 13A illustrate a first step where raw data is read and converted to a pseudo-2D mesh array.

FIG. 13B shows the before and aftereffects of the smoothing process.

FIG. 14 shows a micropore analysis tool comprising an OCT (optical coherence tomography.

FIG. 15 illustrates a screenshot of a pre-planning mode.

FIG. 16 shows a micropore tool including a registration feature.

FIGS. 17 and 18 shows example steps and workflow of a pre-scan visit process.

FIG. 19 shows an example screen shot.

FIG. 20 depicts a block diagram illustrating an example of a computing system.

### DETAILED DESCRIPTION

Systems and methods described herein include a number of aspects which may be usefully employed in combination or separately, and which may be advantageously used to treat a range of disease conditions, both of the eye and other regions of the body. At least some of the examples described in particular detail focus on treatment of conditions of the eye, such as the treatment of age-related glaucoma, cataract formation, and presbyopia, and other age-related ocular diseases such as age-related macular degeneration, or the like.

In general, hardware and software system solutions are presented herein that provide real-time, interactive predictive simulations of the eye (e.g., human eye or animal eye). As described in more detail below, the predictive simulations of an individual's eye can be created using a Finite Element Model (FEM) of ocular structures involved in optical biomechanics, including ocular accommodation. Unlike previous ocular models, the present FEM (finite element model) described herein is a three-dimensional (3D) FEM of the accommodative mechanism, including but not limited to, the anterior human eye that simulates the action of ciliary muscle fiber contraction to predict dynamic deformation of the lens required for accommodative optic power change described herein as “Central Optical Power” (COP). The present FEM further describes the aqueous/vitreous outflow and mechanisms of fluid flow described herein as “Hydrodynamics”. The virtual FEM further describes the posterior eye biomechanics that simulates the actions of the Bruch's membrane, retina, vitreous and lamina cribrosa during the action of pre-stretch, disaccommodation and accommodation of the eye.

More specifically, the disclosed systems and methods include a virtual eye simulation analyzer (VESA) which utilizes virtual eye modeling to create an accurate and realistic virtual interactive model of an individual's eye, and thereafter, provides a 3D FEM based on such model to develop treatment options, predict future states of the eye based on the individual's age, material properties, optical properties, among other physical parameters to enable it to predict effects of various manipulations such as surgical, therapeutic interventions and disease states, etc. The virtual model of the individual's eye therefore holds within its constructs the biometric, mechanical, optical and physical information as an unique 3D-ID for the individual's eye. Moreover, the resulting virtual 3D FEM model, and thus VESA, allows the user (e.g., user, user) to examine the biomechanical characteristics and various physical, optical and physiologic states (e.g., accommodation, disaccommodation, glaucomatous, etc.) of the virtual 3D model, which in turn, allows, for example, the user to more accurately simulate different treatments and determine the treatments effectiveness relative to the real eye of the individual. In other words, VESA provides in silico virtual method to perform a plurality of treatments and manipulations on an individual's virtual eye model to determine, in real time, output information (e.g., scleral notch, ciliary muscle fiber movement, and movement and shape of the lens) that can inform the user of current and future treatment effects, outcomes and probability of success for the individual's eye.

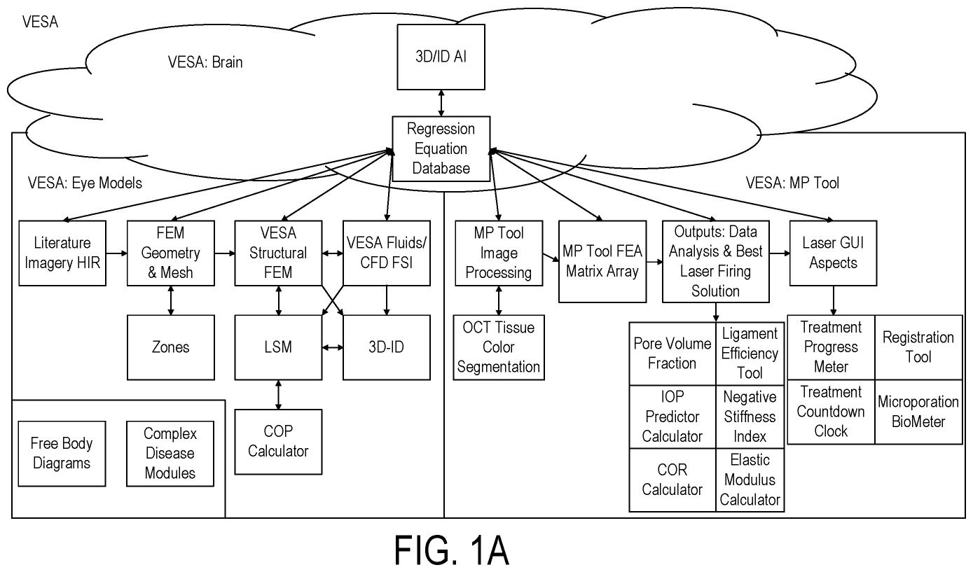

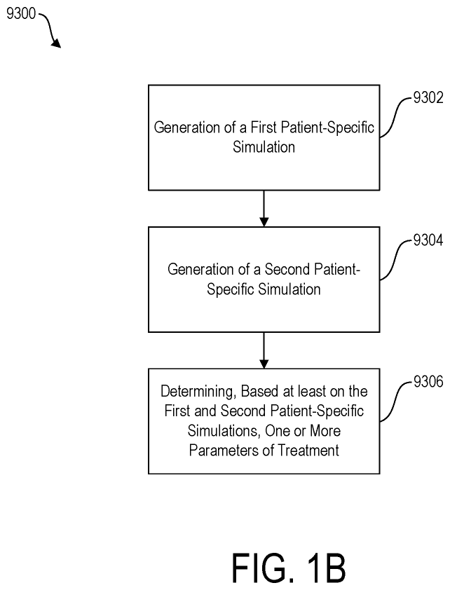

An exemplary chart illustrating VESA, 3D-ID AI, MP Tool, and Regression Equation Database and their relationships to one another are shown in FIG. 1A. While this Figure illustrates VESA separate and apart from the 3D-ID AI, MP Tool, and Regression Equation Database components, such components in essence are part of VESA's ecosystem. As shown in FIG. 1A, VESA includes various elements, as explained in more detail below.

Regression Equation Database: A cloud database configured to store pre-ran and manual individual-specific finite element analysis (FEA) models whether structural, fluids, or from the micropore tool (i.e., captured from clinical trials pre- and post-operative). For example, the input(s) can include individual specific treatment or therapeutic data results and specific eye geometric variables, and the data and regression equation outputs populate the database. Rather than running computationally extensive FEA simulations live during the procedure, this database allows VESA to reference and pull from the pre-built cloud library database of FEA runs to allocate towards each individual, which can then be used to suggest possible pore matrix laser firing solution(s) to arrive at desired results.

Laser Scleral Microporation (LSM) is a type of treatment applied to the human eye that helps to un-crosslink the underlying microfibers in human scleral tissue. A numerical analysis package called the Micropore Tool (MPTool), which is based on ocular rigidity and Newtonian mechanics, is described herein. Using OCT images as the main input data, the MPTool creates data structures that represent the internal and external ocular geometry, all individual pore geometric parameters and pore matrix geometry for every treatment area on both eyes, for a particular individual's eye properties. MP tool further allows the medical technician to experiment with pore depth, diameter, and spacing to effect decrease in biomechanical stiffness or increased compliance in virtual reality so that VESA is enabled to perform numerous simulations in order to recommend the most viable pattern, depth, and location of the pores within the array. The MP tool receives mathematical and physics inputs from the Ligament Efficiency Tool (LET) which is capable of calculating and predicting the strength, flexibility and tissue differential between each pore (i.e., a ligament). Numerous simulations are performed to allow the LEC to suggest the an improved or most ideal pore configuration. The MP Tool, is capable to create the regression equation database of VESA, so that the VESA's FEM(s) can be derived from not only literature imagery but also post-procedure analysis of individuals which can be used to train the neural network of VESA. Hyper-Elastic Image Registration is an image restoration within the 3D FEM inclusive of registration of nonlinear deformations and shape changes. (e.g., during clinical trials, etc.). The micropore tool includes various components. VESA is capable of housing a plurality of customized toolboxes for various surgical and/or therapeutic VR interactions and manipulations. Some preferred embodiments which are currently developed are as follows:
Micropore Tool Artificial Intelligence and Machine Learning Module (AI_ML):AI_ML is a set of routines that pre-process OCT images and prepares the data for further analysis. The AI_ML routines do the following tasks:
   Reads the proprietary binary OCT format.
   Creates several data structures to pre-process the data and represent all quadrants, eyes, and patients in the treatment session. Those data structures include, but are not limited to:
      A meshed micropore tool pore matrix square based on OCT analysis,
      Image color segments,
      Tissue border segmentation,
      Top-to-bottom thickness,
      Individual pore geometry including, but not limited to: 3D coordinates of the pore centroid, pore volume, pore depth, pore diameter, pore volume fraction, pore density,
      Feature edge detection,
      RGB real time biofeedback color recognition depth control feature3.
   Runs several color smoothing and interpolation routines.
   Runs feature recognition to distinguish between sclera top-to-bottom thickness and pore void space.
   Runs Auto-Tissue-Segmentation routines to distinguish between layers in the sclera-conjunctive-choroid geometric complex.
   Runs an edge detection post-processor on the outer boundary of the sclera top layer, and the bottom layer.
   Saves the results of the pre-processing in a second set of data structures that the various downstream tools can read (PVF, NSI, COP, IOP, etc.).
   Makes data ready for consumption of the Micropore Tool, the PVF calculator, NSI calculator.
Micropore Tool Physics Package
   1. Pore Volume Fraction Indicator Tool: A graphical user interface (GUI) evaluation and validation tool that measures the pore volume fraction across a given 1D, 2D or 3D area. Pore volume fraction is variable to scleral thickness by zone, micropore shape, micropore density, and micropore size. The pore volume fraction indicator tool can provide information of the tissue removed except information relative to location.
   2. IOP Predictor: The IOP Calculation Predictor tool is capable of predicting and measuring the change of the intraocular pressure within the eye as it relates to a primary condition of an individual's eye and or as a resultant current or future implication derived from a selected intervention via release and change of hydrostatics and hydrodynamics.
   3. Elastic Modulus Calculator: A processor of the biomechanical and physics data inputs from the BioMeter which takes into consideration the material properties, biomechanics, geometry and biometry of the eye tissues. Applications are for 3 different uses: Pre-treatment planning, peri-treatment evaluation, validation, and post-treatment validation.
   4. Negative Stiffness Index (NSI)/Quotient Calculator(NSQ)/Predictor(NSP): A real time material property & biomechanical property calculator indicating the Negative Stiffness quotient of the biomechanical property/physics resultant change of ocular tissue during and after a treatment,. One preferred embodiment is the nature and biomechanical properties of the scleral tissue before & after LSM treatment. NSI is dynamic and measured real time progressively by treatment progression. In this example, there are four quadrants of the eye treated in the LSM procedure (Q1-Q4) until desired NSQ is met for the designated tissue. Inputs are from the BioMeter, the ligament efficiency calculator (LEC), and the elastic modulus calculator (EMC).
   5. Ligament Efficiency Tool (LET): The physics package behind the micropore tool (MPTool) that determines things such as, but not limited to, pore placement and good surgical judgement.
MPTool—Ocular Rigidity Post-Processing Tools
   Hyper-Elastic Image Restoration: The HIR: is a highly robust numerical simulation of hyperelasticity as a regularizer and introduces a new and stable numerical implementation of highly nonlinear deformations,
   OCT Tissue/Color Segmentation: Depth Sensing Tool AI auto-segmentation software.
      that is displayed over OCT scans.
      It creates an augmented reality view of the biometry of the tissue layers of the eye.
      The Overlay demonstrates key tissues, such as, but not limited to, the cornea, sclera, conjunctiva, zonnules, ciliary muscle fiber, and tenon's capsule while correlating RGB data base colors to real time interactive tissue identification.
Micropore Tool Simulation Package: The Simulation package consists of a toolset that allows the user to use the same pore matrix analysis tools on simulated, rather than real, pores, pore matrices, and human eyes. The toolset includes:
   Several sets of FEA runs that fully describe the individual patients' eye and possible LSM outcomes,
   Databases, to house the FEA runs and run regression algorithms on those data,
   Live Finite Element runs, contemporaneously done with the LSM treatment,
   Other empirically derived numerical methods like moment of inertia.

FEM Geometry and Mesh: Commercial FEM biomechanics simulation software is used to generate the geometry and mesh of the complex human eye model of accommodation. The input is geometrical data obtained from, for example, published literature (e.g., literature imagery, Hyperelastic Image Restoration (HIR)) and/or the regression equation database, ultrasound biomicroscopy (UBM), optical coherence tomography (OCT), scanning electron microscopy (SEM), and histology. The analysis of this FEM Geometry and Mesh involves a geometry built in SpaceClaim and meshed in ANSYS. Boundary conditions, loads, elastic supports, neo-hookean material properties, stresses, tensions, forces, moments, displacements, etc. are thus applied within ANSYS.
Zones: As discussed in more detail below, the FEM geometry and mesh of the eye are built with zones, which are built with distinct pore matrix firing regions that are chosen based on the geometric locations of specific anatomical landmarks found in literature and/or the regression equation database.

VESA structural FEM: The eye model of accommodation built within ANSYS. The input includes, but not limited to, the FEM Geometry and Mesh, and the outputs include, but are not limited to, simulation(s) of multiple age eye models of accommodation, which are improved accommodations that are based on change in diopters value of the individual's vision captured from the central optical power (COP) calculation.
Laser Scleral Microporation: See above description.
Central Optical Power Calculator: This calculator relates to the COP of the crystalline lens. Given that the deformation of the crystalline lens changes its optical power in diopters (D), this calculator is designed to calculate the free body forces of the lens. The inputs can include, for example, the data from simulations of accommodation before and after the LSM procedure. Expanded formulas are presented as an alternative to this calculation by using Ray Tracing calculated measurements as a direct input to COP. This invention is novel for this reason.
Example Embodiment is the Len's formula derived by Ray tracing; whereas, the lens shape is determined by Finite Element Analysis. The shapes are then exported to a software process or other process which traces the paths of rays in both accommodated and non-accommodated state. The COP is then determined by using:

$$C O P = \frac{1}{f Accommodated} - \frac{1}{f Non - Accomodated}$$

VESA Fluids Simulation: Computational Fluid Dynamics (CFD) & Fluid/Solid Interaction (FSI): It is similar to the VESA structural FEM, but here, the eye model of accommodation concept built within ANSYS also captures dynamic fluids, inflows, and outflows. As such, the input includes, but not limited to the same geometrical/mesh data from the FEM Geometry and Mesh with the addition of added geometrical fluid pathways to allocate for specified fluid movement based on, for example, clinical studies and literature Similar to the VESA Structural FEM, the outputs here include, but are not limited to, simulation(s) of multiple age eye models of accommodation, which are improved accommodations that are based on change in diopters value of the individual's vision captured from the central optical power (COP) calculation. Further, the incorporation of fluid in an FSI model can make VESA's FEM much more accurate to the reality of how the human eye works. This is because VESA's structural FEM includes assumptions made to account for any lack of dynamic fluids within the FEM. For example, an assumption can be made that the fluid is present and incompressible, i.e. the interior volume remains constant during accommodation.

3D-ID: The input(s) can include individual-specified VESA Structural/Fluids models (FSI) that are analyzed through accommodation simulations before and after the LSM procedure. As a result, the output is accommodative improvement based on change in diopters value of the individual's vision captured from the central optical power calculation. Thus, VESA is capable of being used as a virtual platform to import individual-specific eye related data thus making any individual's eye into their own VESA structural and/or fluids model. The 3D-ID also include the individual specific geometry, stored in a CAD file or some similar database. The 3D-ID includes all individual specific metadata that is collected during the treatment process such as name, age, sex, and ethnicity.

3D-ID AI: 3D-ID AI is a machine learning based analysis engine communicatively coupled with VESA, the MP Tool, and the Regression Equation Database. 3D-ID is further derived from the ‘age progression’ formula which accounts for a normative progressive change in material properties and biometry changes which occur with age. 3D-ID AI may apply one or more off-the-shelf machine learning or bespoke regression models deep learning or regression trained to determine, based on real time FEA simulations performed by VESA and/or pre-existing individual-specific FEA models from the Regression Equation Database, one or more individual specific treatment parameters. The machine learning models associated with 3D-ID AI may include regression models (and/or other types of machine learning models) that have been trained based on simulated and/or clinical data of various treatment parameters, conditions and treatment outcomes (e.g., accommodation improvement based on change in diopters value of the individual's vision captured from the central optical power calculation). Through this training, the machine learning models of 3D-ID AI learns the relationship between different combinations of conditions of the eye, treatment parameters and the corresponding treatment outcomes, and progressive trends in responses to treatments such as age-related changes in material properties over time. Moreover, in some cases, the 3D-ID AI machine learning models may be trained to take into account additional individual data, such as demographic information. Accordingly, 3D-ID AI is capable of determining, in silico, precise treatment parameters that are suitable for each individual prior to undertaking any in vivo procedures. For example, in some example embodiments, the output of 3D-ID AI may be a dosage for a particular individual, which includes one or more of pore matrix configurations such as a quantity of matrices, a placement of the matrices, an overall shape of each matrix, an overall dimension of each matrix, a quantity of pores in each matrix, a distribution of pores in each matrix, a depth of pores in each matrix, and/or the like. For example, another output of 3D-ID may be a predication of dosage of treatment over time as well as number of treatments required over a period of time. For example, another output of 3D-ID may be a suggestion of a type of intraocular lens that may be more suitable or desirable for a particular outcome of an individual's eye given the conditions of that individual's eye for the capability of achieving COP in various focal points. For example, another output of 3D-ID may be a suggestion of a type of Minimally Invasive Glaucoma Surgery (MIGS) implant that may be more suitable or desirable for a particular outcome of an individual's eye given the conditions of that individual's disease state. A plurality of virtual treatment options can be presented to VESA for solving of a particular individual's eye via the 3D-ID plug in.

It should be appreciated that the 3D-ID AI machine learning models may undergo additional and, in some cases, continuous training. For example, for a set of treatment parameters output by 3D-ID AI, 3D-ID AI may receive feedback from a user administering the treatment, including real time adjustments made to one or more treatment parameters. The 3D-ID AI machine learning models may be updated based on these additional data points, thus allowing the 3D-ID AI machine learning models to learn additional and/or more nuanced relationships between treatment parameters, treatment outcomes, and other individual specific data (e.g., demographics).

VESA can include additional elements, such as, but not limited to:
Registration Tool: Micropore Registration Tool (MIRT): Provides information regarding the dynamic location of the pore or a spot, as well as the pattern by utilizing Matrix Registration Tool (MART).
Microporation BioMeter: Individualized Biomechanical/Physics inputs to the Elastic modulator Calculator (EMC) & 3D-ID VESA components: Age Related mpa, Depth, PVF, Bulk Density (aka Regional Stiffness), Density, Pore Size, and Pore Shape (Square).
Treatment Progress Meter: Progressive completion meter regarding treatment progress and synchronized to a time clock for countdown of each quadrant. Done by quadrant with the countdown incorporated.
Treatment Countdown Clock: A GUI treatment countdown clock.

1Hyperelastic Image Registration (HIR) for Analysis of UBM: Reverse imaging of dynamic biological movements translated into a 3D FEM.
Artificial Intelligence/Neural Network: Statistical and/or heuristic approaches to implementing a decision engine for the VESA system.
Hoop Stress Coordinate Map: Real-time physics calculation/depth calculation Global Positioning System (GPS). Hoop stress is the stress that occurs along the pipe's circumference when pressure is applied. Hoop stress acts perpendicular to the axial direction. Hoop stresses are tensile and generated to resist the bursting effect that results from the application of pressure. HSCM demonstrates a diagram of the various Hoops that exist on a sphere that conform to annular structure theory. Used to construct the Hoop strain constitutive formulas. HSCM has further utility in developing an infinite number of patterns of treatments based on the nodes and array of nodes that can be aligned for ideal treatment for example with LSM so that the pores are created with specific purpose.
4D HyperShere Coordinate Scanning & Plenary Mapping (3D ID: Is a Global Positioning System (GPS) Post Processing Tool to VESA FEM): Virtual OCT post processing scanner. 4D HCSPM can be utilized as a training database for VESA.
Resultant Force Calculator: Calculates the free body forces & moments on the lens capsule of the eye. Used to determine the reaction velocities and direction of the lens during accommodation.
Spherical Stiffness Indicator: Stiffness calculated on a 3D shell that adheres to a spherical coordinate system. Hoop stress and strain are emphasized.
Coefficient of Ocular Rigidity: A mathematical formula predicting a constant as a macroscopic parameter characterizing the relationship between pressure and volume changes in the human eye.
Dosage Regulator: Dosage regulator capability to control, predict, administer, calculate, and report, dose of surgical, chemical biochemical, therapeutic, physical, fluidic, geometrical, biological and/or biomechanical interventions in virtual reality.

Further, additional information on certain elements of VESA can be found in U.S. Pat. No. 11,071,450 and U.S. Patent Publication No. 2020/0185106, which are each incorporated herein by reference in their entirety.

VESA is configured to enable applications not otherwise possible with conventional eye modeling systems and methods. For example, such applications can include the affirmation and visualization of cause-and-effect relationships of accommodative function intuited from correlated observations of ocular geometry, material properties, physical forces and dynamic shape change. This affirmation can be effected by VESA through the FEM prediction of the excursion (dynamic 3D shape change) of the ciliary muscle fiber movements and forces during all phases of accommodation from far to near and near to far focus OR from prestretch, to disaccommodation to accommodation as well as maximum accommodation to disaccommodation to pre-stretch. These phases, movements and forces are a result of the combination of elastic stored energy in the BMCC, specified ciliary muscle fiber architecture\specified zonular geometry and architecture and their respective prescribed activation. The deformation (dynamic 3D shape change) of the lens from pre-stretch, accommodation to a disaccommodated state results from the combination of specified zonular geometry, capsular strain, and prescribed tension, and/or the deformation of the lens during accommodation resulting from excursion of the ciliary muscle fiber. The modeling component being configured to determine a three-dimensional finite element model of the whole eye from the three-dimensional structural image and at least one biomechanical property of the eye. The modeling component is configured to provide a three-dimensional finite element model representing the whole eye after the therapeutic procedure, the system further comprising a user interface configured to accept input from a clinician defining at least a type and location of the therapeutic procedure. The feature extractor is configured to extract the at least one feature of the plurality of features from the three-dimensional finite element model representing the whole eye after the therapeutic procedure, the ectasia evaluation component being configured to calculate a parameter representing an expected risk of ectasia to the patient given a therapeutic procedure having the associated type and location. The ectasia evaluation component being configured to perform a sensitivity analysis on at least one feature, such that a magnitude of an impact of the value of the at least one feature on the at least one parameter can be determined. The extracted at least one feature represents one of a geometric or biomechanical characteristic of the eye. The modeling component is configured to provide a three-dimensional finite element model representing the whole eye with a load applied to the eye, the system further comprising a user interface configured to accept input from a clinician defining at least a magnitude and location of the load. The modeling component is configured to determine a finite element model of the whole eye from the three-dimensional structural image and at least one biomechanical property of the eye. The objective function can be a function of at least one of a strain value of the eye and a stress value of the eye. The objective function can be a function of at least one measure of refractive outcome. The modeling component can determine the representation of the whole eye as a virtual model comprising a set of parameters extracted from evaluating a statistical model according to the three-dimensional structural image and at least one biomechanical property of the eye

Another application can include estimation of aspects of accommodative function that are currently experimentally unmeasurable. By way of example, this estimation can be carried out through VESA's FEM by analyzing the biomechanical effects of ciliary muscle fiber fiber sections, perform simulations where each ciliary muscle fiber fiber section is activated in isolation to quantify its unique contribution to the deformation of the lens, and/or perform simulations where each zonular division is tensioned in isolation to quantify its unique contribution to the deformation of the lens. Any anatomical component of the accommodative system can be run in isolation to determine its unique contribution to the lens shape change output in virtual reality which is not possible to investigate in vivo or ex vivo. An infinite number of manipulations of geometry, activation, physical properties, material properties and biomechanical relationships and interactions are possible with VESA which are not possible in vivo.

Further, as noted above, the VESA provides the ability for in silico experimentation which allows a user (e.g., doctor or surgeon) to understand pathophysiology of accommodative dysfunction and determine effective methods of treatment through performing various simulations and “What if” scenarios For example, simulations where ciliary muscle fiber section activations are altered to reflect changes due to measured atrophy or connective tissue infiltration in older adults with presbyopia (age-related accommodative dysfunction) to compare predicted accommodative lens response, and/or simulations where material property of passive structures (e.g., lens components, scleral zones) are changed in isolation to reflect measured stiffness changes in older adults with presbyopia to compare predicted accommodative lens response/COP, and/or measured stiffness changes due to therapeutic interventions (e.g., intraocular lens replacement, scleral microporation) to compare predicted accommodative lens response.

In one exemplary VESA embodiment, the FEM identifies, for each individual, precise ciliary muscle fiber sections that require the placement of one or more pores in different sections (e.g., scleral zones) of the eye. This VESA FEM allows for the isolation of individual ciliary muscle fiber sections in silico. For example, each simulation can isolate at least one ciliary muscle fiber fiber section (or some combination of muscle fiber sections) so at the conclusion of an x-quantity of simulations, the user can determine where and how many pores need to be added to the eye. In other words, VESA is configured not to just model the individual's eye as a whole but also in discrete sections to help determine the desirable location(s) of the one or more pores to be created in the eye, for example, via laser scleral microporation (LSM). Moreover, the VESA FEM is capable of isolating individual ciliary muscle fiber sections in silico. VESA is capable to perform these isolated analyses for any component of the anatomical system of the eye such as the elastic movements of the BMCC apparatus.

FIG. 1B illustrates and exemplary process flow chart 9300 that generally includes generation of a first individual-specific simulation 9302, generation of a second individual-specific simulation 9304, and determining, based at least on the first individual-specific simulation and the second individual-specific simulation, one or more parameters of a treatment 9306. In one exemplary embodiment, the first individual specific simulation 9302 can include activation of a first ciliary muscle fiber section of the individual's eye to determine a first contribution of the first ciliary muscle fiber section to a deformation of a lens of the individual's eye, the second individual specific simulation 9304 can include activation of a second ciliary muscle fiber section of the individual's eye to determine a second contribution of the second ciliary muscle fiber section to the deformation of the lens, and the one or more parameters of a treatment 9306 can include the placement of one or more pores in the first ciliary muscle fiber section and/or the second ciliary muscle fiber section. In some example embodiments, the one or more parameters of the treatment 9306 may be determined by applying the machine learning based analysis of 3D-ID AI. Examples of treatment parameters, which are precisely determined to be optimal for each individual, may include a quantity of matrices, a placement of the matrices, an overall shape of each matrix, an overall dimension of each matrix, a quantity of pores in each matrix, a distribution of pores in each matrix, a depth of pores in each matrix, and/or the like. Other anatomical structures beyond the ciliary muscle fibers are within the scope of this disclosure. For example, the specific stimulations, such as the first, second and/or third specific stimulations can comprise activation of one or more ciliary zonules or any of a ring of fibrous strands forming a zonule that connects the ciliary body with the crystalline lens of the eye. Such fibers are sometimes collectively referred to as the suspensory ligaments of the lens. The system can consider, model, or otherwise take into account zonular forces and anatomical response. In this response, the zonules can be divided into separate regions or groups based on anatomical position, force applied by the zonule, or any other factor.

More specifically, the FEM's identification of desirable location(s) of the one or more pores is based on VESA's components and capabilities, such as, but not limited to, a detailed representation of ciliary muscle fiber architecture and contractile behavior, taking into account pre-tension applied to the lens via zonular structures, and a discrete 3D geometric representation and independent material specification of passive structures of the eye that mechanically interact during accommodative function. These passive structures can include, but are not limited to, lenticular structures (e.g., lens capsule, lens cortex, lens nucleus), scleral zones (e.g., Zone 0, Zone 1, Zone 2, Zone 3, Zone 4, and beyond), posterior sclera, and/or extralenticular structures (e.g., cornea, scleral spur, zonnules, lamellae layer, choroid, bruch's membrane vitreous membrane).

By way of example, the detailed representation of ciliary muscle fiber architecture and contractile behavior is generated based on, but not limited to, discretized 3D geometry of three sections of muscle fibers, fiber directions assigned to muscle geometry to represent architecture of each section, constitutive law defining material behavior where stress in the fiber direction scales with activation level, and time varying prescription of activation level in each section that results in dynamic contraction. Similarly, the pre-tension applied to the lens via zonular structures is generated based on discretized 3D geometry of seven divisions of zonules, represented as 3D sheets extending between attachment points, fiber directions assigned to zonule geometry to represent fibers within each sheet, constitutive law defining material behavior where stress in the fiber direction scales with tension level, and time varying prescription of tension level in each division that results in dynamic tensioning.

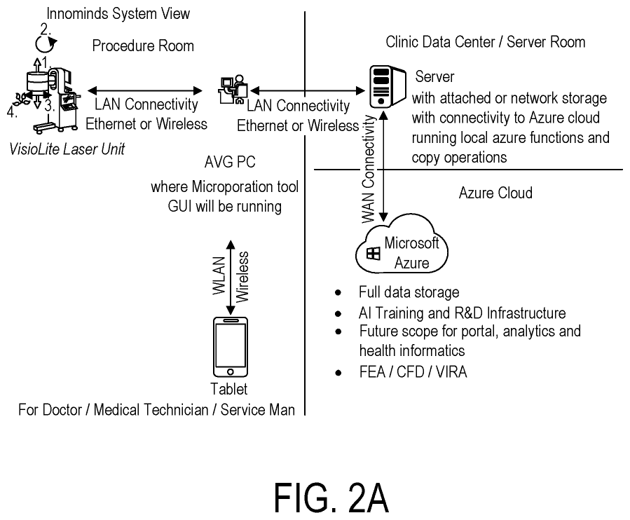

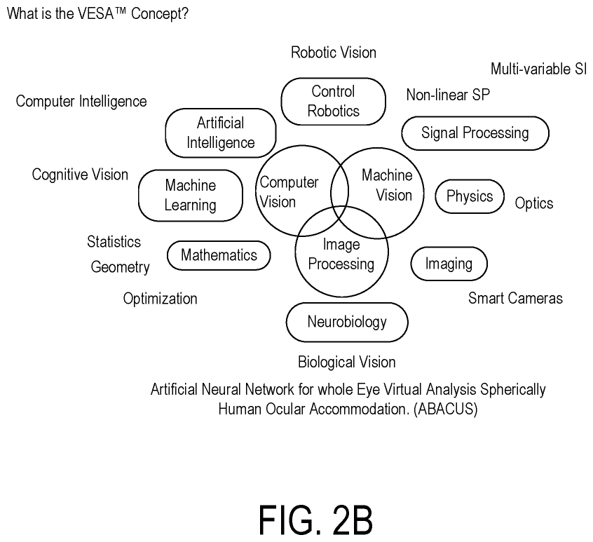

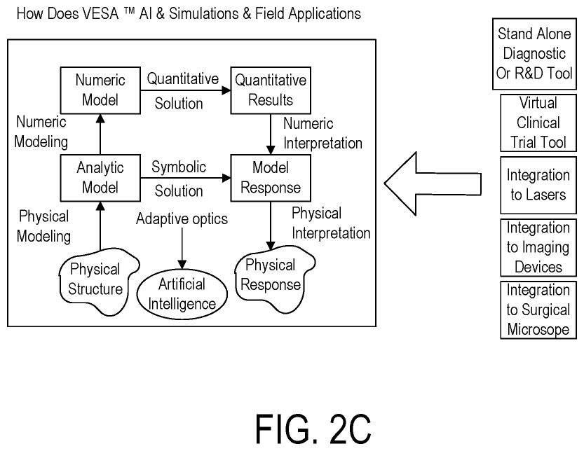

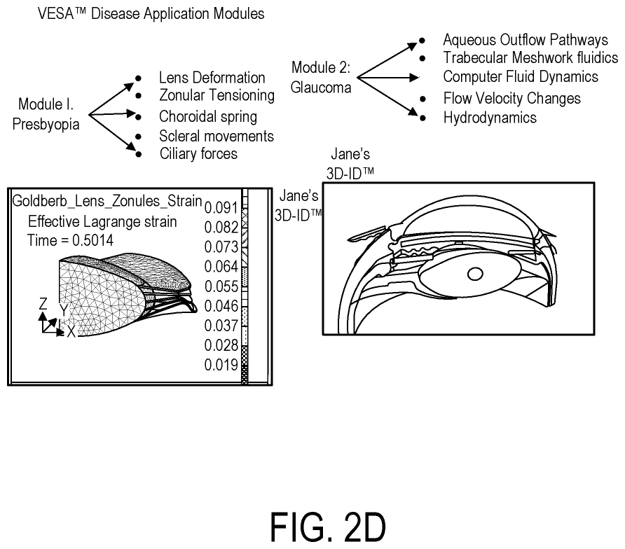

FIG. 2A illustrates a VESA ecosystem having various hardware/software systems residing in the procedure room, the cloud, and/or database. FIG. 2B illustrates the various technologies and tools that make up the backend services of the VESA ecosystem. The backend services can reside on one or more computing systems such as the AVG PC unit or on the cloud. As shown in FIG. 2B, VESA includes the application of AI, neural networks, machine learning, signal processing, mathematics, neurobiology, physics, imaging, and robotics.

FIG. 2C illustrates a plurality of sub-systems of the VESA ecosystem that includes standalone diagnostic tools, clinical trial tools, lasers, imaging devices, AI engines, etc. FIG. 2D illustrates two of the many disease application modules. Module 1 is for treating presbyopia and module 2 is for treating glaucoma. For module 1, the modeling of lens deformation, zonular tensioning, choroidal spring properties, scleral movement, and ciliary forces are important. For module 2, tool such as FEA and/or computational fluid dynamic (CFD) can be used to model at least the following properties of the eye: aqueous outflow pathways, trabecular meshwork fluidics, flow velocity changes, and hydrodynamics. In some embodiments, VESA can measure intraocular pressure (TOP) of the eye using CFD and/or constitutive calculation. VESA can employ FEA to calculate or simulate the deflected shape of the IRIS.

VESA can also perform simulation on pipe flow to predict flow volumes in the eye using CFD. In some embodiments, the CFD model can simulate/model pump efficiency, constricted outflow, and ambient pressures. This CFD is also capable in VESA to demonstrate the Fluid Structure interactions or FSI of the particles and liquids in the aqueous outflow system as well as the outflow pathways both under normal and perturbed conditions. Modeling the geometry from the SIMSCALE Forum can be used. A 3.6 mL/day of flow rate of aqueous formation can be used as input to the CFD model. Porous media can be used in the model. ANSYS Fluent was used to develop the model. Other modeling software can also be used. In modeling the flow volume, one or more of the following can be assumed:
The modified Goldmann equation summa_x0002_rizes the relationship between many of these factors and the intraocular pressure (TOP) in the undisturbed eye,
P0=(F−U)/C+Pv,
where P0 is the IOP in mm Hg,
F is the rate of aqueous formation in microliters per minute (μL/min),
U is the rate of aqueous humor drainage through the pressure-insensitive uveo_x0002_scleral pathway in microliters per minute (μL/min),
C is the facility of outflow through the pressure-sensitive trabecular pathway in microliters per minute per mm Hg (μL/min/.
mm Hg), and Pv is the episcleral venous pressure in mm Hg. Resistance to outflow (R) is the inverse of facility (C).

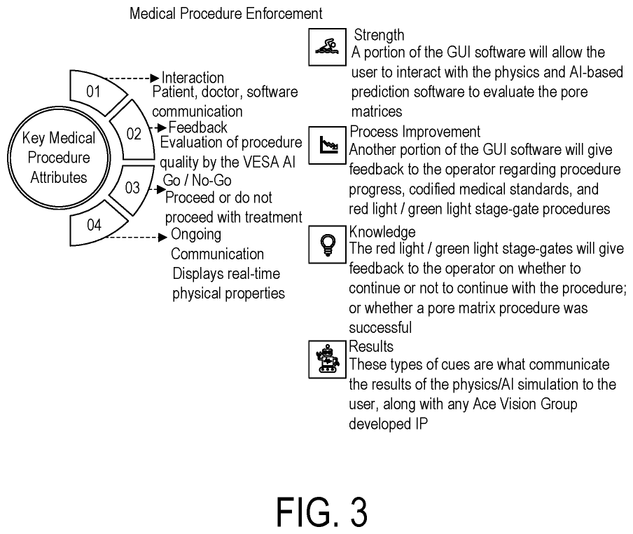

FIG. 3 illustrates how VESA can contribute at various stages of a key medical procedure. As shown, VESA can add many benefits in all stages of the medical procedure by providing powerful AI-based predictive analytics (e.g., predictive software) and improving processes and enhance knowledge.

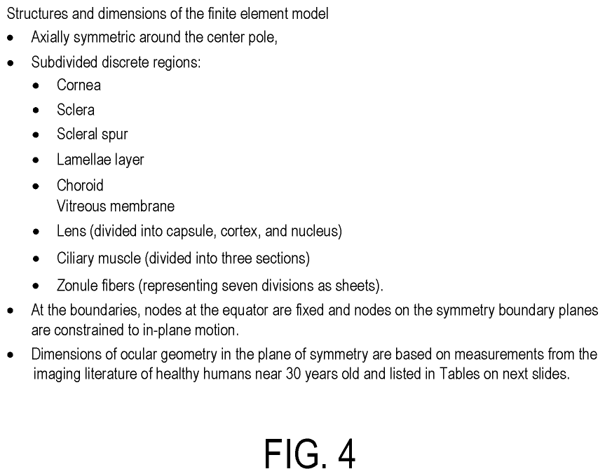

As mentioned, VESA includes one or more finite element models of an individual eye. There can be one or more assumptions for the overall eye model. For example, a portion of the sclera can be segmented into 5 different zones for modeling. Each zone can be additionally defined by its own material and biomechanics properties. FIG. 4 illustrates the structures, various measurable variables, and boundary conditions that can be used to generate the finite element model of the eye. As noted, the zonules of the eye are within scope of this disclosure.

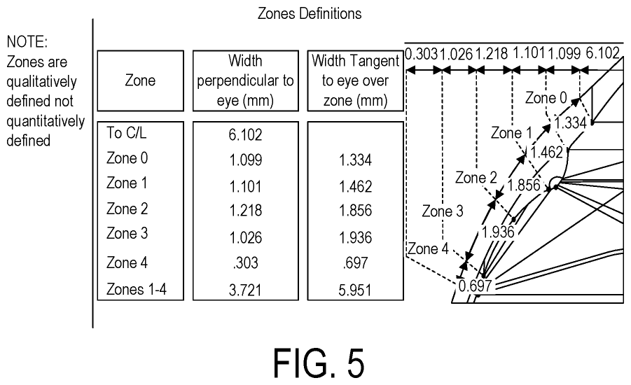

FIG. 5 illustrates exemplary zone definitions that can be used to generate the finite element model. In some embodiments, the zone assumptions are:
Different sections of the ciliary muscle fiber blend together so there are no references that provide specific dimensions.
The zones are perpendicular to the outer scleral surface. So Zone 0 is the region between a line starting at the inner point of the spur and another line starting at the inner point on the limbus.
The model is built with distinct regions and the boundaries were chosen to recapitulate the descriptions of each section in the literature. The boundaries of each zone are based on the geometric locations of specific anatomical landmarks in the 30-year-old eye model.
The description of the boundaries with respect to specific anatomical landmarks will vary somewhat for each subject.

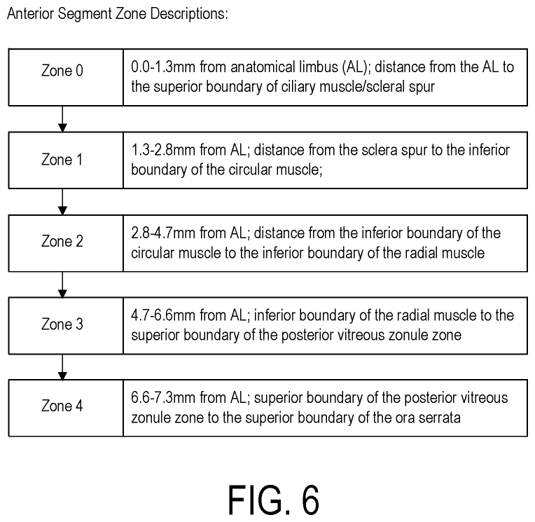

FIG. 6 illustrates examples of each of the zone description or anatomical location with respect one or more areas of the eye.

VESA can take into account the properties of at least one or more of the following non-limiting elements:
Corneoscleral Junction
Cornea (License)
Lens in Detail
Lens Capsule in Detail
Weigert's Ligament
Anterior Angle structures
Retina
Choroid
Bruch's membrane
Hyaloid membrane
Aqueous
Vitreous
Eye Shape
Axial Length
Ciliary Body, all muscle fiber directions
Ciliary Apex
Optotype
IRIS (pupil)
Scleral biomechanical, elastic properties
Scleral—Annulus of Zinn Junction
Muscular attachments of all ciliary muscle fibers
Lamina cribrosa
All Zonnular attachments in detail
Lens Capsule
Scleral Spur
Schlemm's canal
Subchoroidal space
Subchoroidal lamina
Trabecular meshwork

In some embodiments, the fluid-solid interaction solver can model the trabecular meshwork using software such as, but not limited to, Ansys Fluent. The trabecular meshwork can include the following anatomical features:
Corneoscleral—Contains a large amount of elastin, arranged as a series of thin, flat, perforated sheets arranged in a laminar pattern; considered the ciliary muscle fiber tendon.
Juxtacanalicular—Juxtacanalicular tissue (also known as the cribriform meshwork)—Lies immediately adjacent to Schlemm's canal, composed of connective tissue ground substance full of glycoaminoglycans and glycoproteins. This thin strip of tissue is covered by a monolayer of endothelial cells.
Schlemm's canal is a circular lymphatic-like vessel in the eye that collects aqueous humor from the anterior chamber and delivers it into the episcleral blood vessels via aqueous veins.
The canal is essentially an endothelium-lined tube, resembling that of a lymphatic vessel. On the inside of the canal, nearest to the aqueous humor, it is covered by the trabecular meshwork; this region makes the greatest contribution to outflow resistance of the aqueous humor.
Conventionally, the canal has been considered a blood vessel, but studies published in 2014 showed that the molecular identity of Schlemm's canal is very similar to the one of lymphatic vasculature.
Major Arterial Circle of Iris—A vascular circle located in the anterior part of the ciliary body near the root of the iris. It is formed by the anastomosis of the two long posterior ciliary arteries and the seven anterior ciliary arteries. It supplies the iris, the ciliary processes and the anterior choroid.
Ciliary Process Capillaries—These capillaries supply blood to surrounding tissues.
The tributaries of the eye including but not limited to: Anterior and posterior ciliary veins. The two superior two vorticose veins (vortex veins) from the upper half of the eye. Episcleral veins from the eyelids and conjunctiva. The central retinal vein. The lacrimal vein.
Collector Channels.

In some embodiments, the meshwork can be segmented into several parts, each with characteristically different structures. They can include, but not limited to:
Inner uveal meshwork—Closest to the anterior chamber angle, contains thin cord-like trabeculae, orientated predominantly in a radial fashion, enclosing trabeculae spaces larger than the corneoscleral meshwork.
Corneoscleral meshwork—Contains a large amount of elastin, arranged as a series of thin, flat, perforated sheets arranged in a laminar pattern; considered the ciliary muscle fiber tendon.
Juxtacanalicular tissue (also known as the cribriform meshwork)—Lies immediately adjacent to Schlemm's canal, composed of connective tissue ground substance full of glycoaminoglycans and glycoproteins. This thin strip of tissue is covered by a monolayer of endothelial cells.
   The trabecular meshwork is assisted to a small degree in the drainage of aqueous humour by a second outflow pathway, the uveo-scleral pathway (5-10% of outflow occurs this way). The uveo-scleral pathway is increased with the use of glaucoma drugs such as prostaglandins.
   The trabecular meshwork had previously been thought to arise from a point (apex) corresponding to the termination of the DM (Schwalbe's line) however it is now considered to extend into the cornea, forming the Dua's layer.
Schwalbe's line is the anatomical line found on the interior surface of the eye's cornea, and delineates the outer limit of the corneal endothelium layer. Specifically, it represents the termination of Descemet's membrane. In many cases it can be seen via gonioscopy.
   Some evidence suggests that the corneal endothelium actually possesses stem cells that can produce endothelial cells, especially after injury, albeit on a limited scale.
   Collector channels are endothelial-lined openings that originate in the outer wall of Schlemm's canal and act as conduits to pass aqueous fluid to intrascleral and episcleral vessels leading to the venous system. The number of collector channels varies in humans, but generally is in the range of 24 to 35 per eye, 8-10 and are distributed in a random manner around the circumference of Schlemm's canal outer wall.
Episcleral Vein—The aqueous humour exits the eye through the trabecular meshwork into Schlemm's canal (a channel at the limbus, i.e., the joining point of the cornea and sclera, which encircles the cornea) It flows through 25-30 collector canals into the episcleral vein.
Major Arterial Circle of Iris
   A vascular circle located in the anterior part of the ciliary body near the root of the iris. It is formed by the anastomosis of the two long posterior ciliary arteries and the seven anterior ciliary arteries. It supplies the iris, the ciliary processes and the anterior choroid.
The conjunctiva is a tissue that lines the inside of the eyelids and covers the sclera (the white of the eye). It is composed of unkeratinized, stratified squamous epithelium with goblet cells, and stratified columnar epithelium.

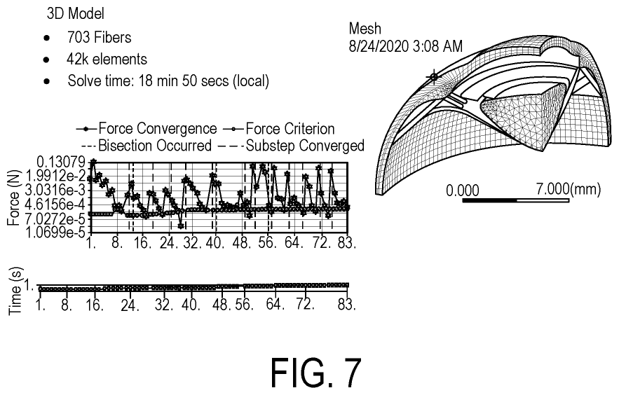

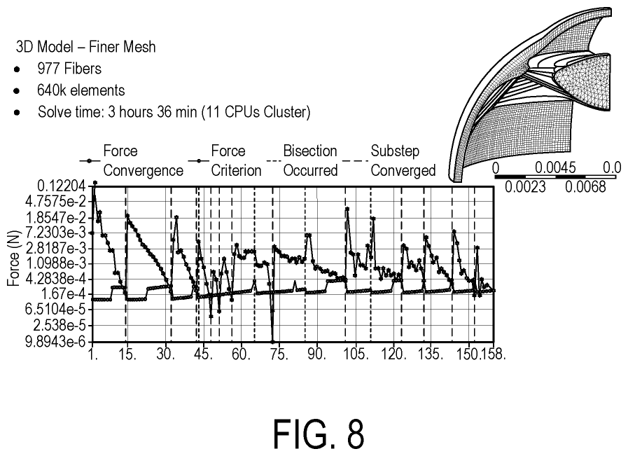

FIG. 7 illustrates an example 3D model generated by VESA. The 3D model is a FEM consisting of 703 fibers and 42 thousands elements. It should be noted more or less fibers and mesh elements can be used. For example, FIG. 8 illustrates a FEM created using more fibers and elements, which would increase the computation time.

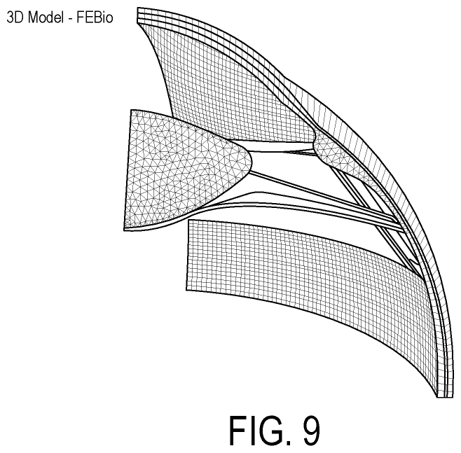

FIG. 9 illustrates an example mesh network of a 3D model of an eye. Additionally, FIG. 9 shows the simulated accommodation of zonules and muscles within the eye and further illustrates the approximation of the zonules as springs for the purpose of generating the FEM. Once the mesh model is generated, the model can be imported into FEBio.

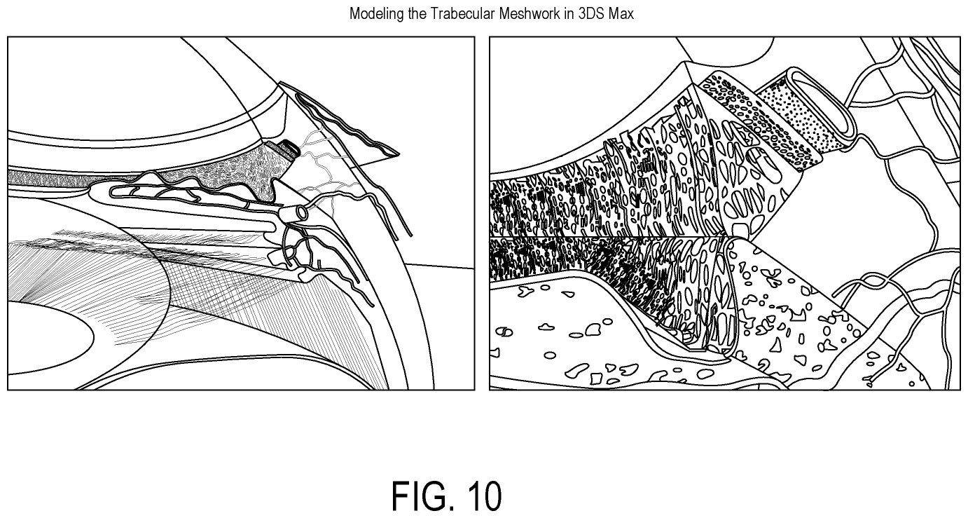

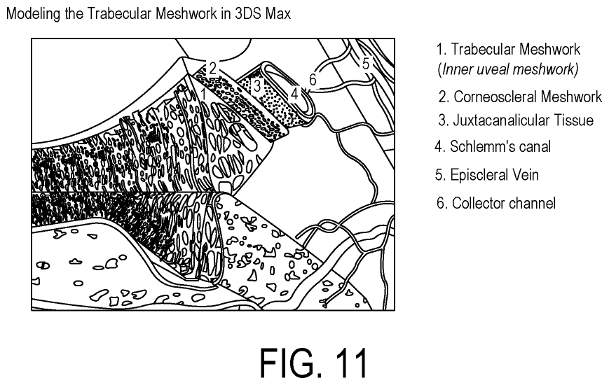

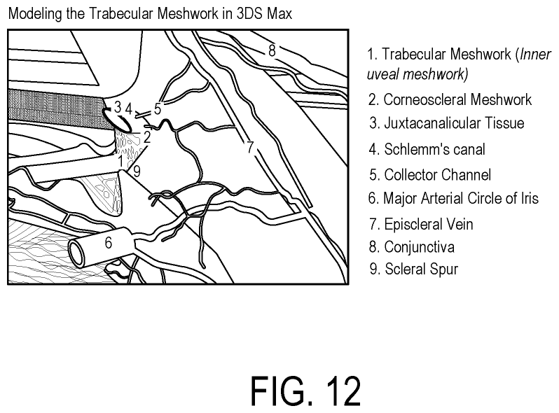

FIGS. 10-12 illustrates an example anatomy model used by VESA to generate the trabecular meshwork. For example, the material properties of each feature (e.g., Schlemm's canal, episcleral vein, conjunctiva) can be assigned to one or more mesh elements associated with the respective feature.

In some embodiments, VESA can include a micropore analysis tool that is configured to analyze and determine the characteristic of a micropore array edged onto the individual's eye. Some features of the micropore analysis tool are listed below. The list is non-exhaustive.

The Micropore Tool (MPTool) micropore analysis tool (or simply “tool”) can arrange xyz data in grid or mesh configuration and display data in a color coded fashion according to the Z value. The Z value is the height coordinate and is a conversion of the grayscale value in the image.

The tool can employ a cell smoothing routine. The tool can implement an edge detection algorithm to find the edges of each pore. Examples of and criteria used to find the edges can be found in this document. Using the edge detection algorithm, a second copy of the grid can be created, with the values not found to be inside a pore discarded. A square box can be drawn around each pore, and these dimensions reported. The edge found in the previous step can be used to draw this box. The height values inside this box can be used to report 1 characteristic height for each pore.

There is now described a process for using the micropore analysis tool to determine characteristics , quantification, and qualification of the micropore array such as, but not limited to, ligament efficiency, pore thickness, and pore height. Using the characteristic height data for the pore grid, the tool can employ the ASME ligament efficiency principals to evaluate the change in bulk pore value of the overall patch imparted by the pore patch.

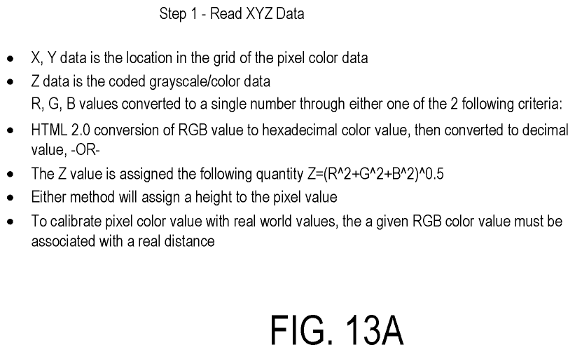

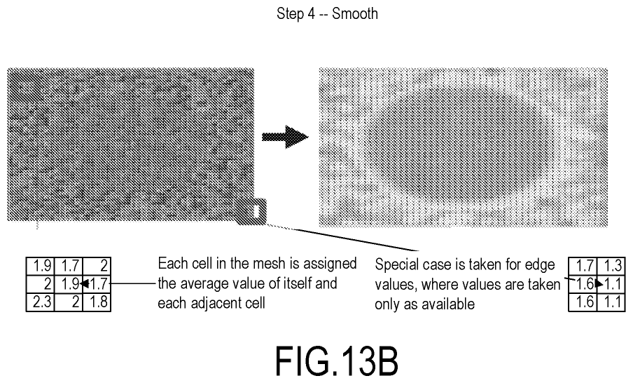

FIG. 13A illustrate a first step where raw data is read and converted to a pseudo-2D mesh array. At step 2 (not shown), the tool is configured to determine the width of the pore block. This can be inputted manually by the user. The width of the pore block can be the average width of pores in the array. Alternatively, the width of the pore block can be programmatically determined using various algorithms and/or computer vision analysis tools.

Next, a color grid of each pore is determined. This eventually creates a color grid representative of a matrix having a plurality of pores. For example, a 5×5 matrix.

Next, the grid is smoothened to smooth out outlier color values that are most likely wrong such as the one indicated by the arrow, which should have a green shade rather than an orange/red shade. Smoothing out the outliers will help the edged detection process. FIG. 13B shows the before and after effects of the smoothing process.

Next, the tool cleans the whitespace. This step can include determining the linear average of the entire pore block (non-zero values); zero out all cells less than 105% of the linear average of the pore block (Note: the 105% qualifier is an engineering judgement. It may have to be adjusted based up the available data); and recolor the data.

Next, the following actions occur:
Assume the remaining non-zero cells to be contiguous as well as generally square in shape (engineering judgement).
Determine the linear average of the remaining non-zero cells. The reported depth of the pore cylinder” will be 80%-90% of this value. This is the characteristic height of the pore.

Lastly, the characteristics of each pore are tabulated and recorded and registered.

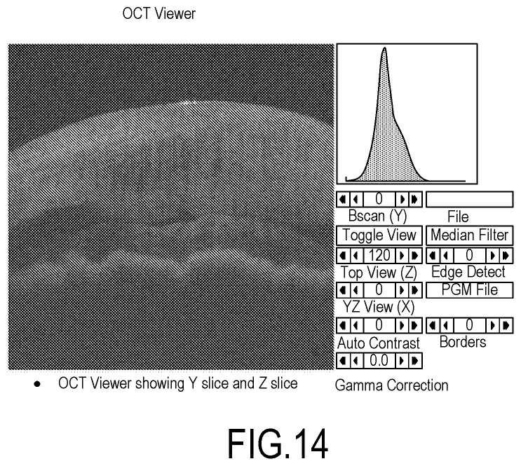

The micropore analysis tool can be an OCT (optical coherence tomography) viewer configured to generate images of the micropore(s) at various views (e.g., X slice, Y slice, Z slice) to measure certain properties of the micropore such as, but not limited to, depth, width, edge, size, shape, coordinates, diameter, distances to adjacent pores, as shown in FIG. 14. The micropore analysis tool can include a plurality of AI (e.g., neural networks) engines trained to perform edge detection, measure diameter, measure depth, layers (e.g., tissue segmentation), etc.

Once the FEM or virtual eye model is created, it can be used to perform additional modeling (e.g., sub-modeling, predictive modeling). The goal of sub-modeling is to determine the new biomechanical properties of the sclera after the surgery. Some of the assumptions of the model are:
The initial sclera is a homogenous or heterogeneous material.
The properties of the sclera can be taken from experimentations and/or accommodation FEM data of previous modeling work. (e.g. Young's Modulus=1.62 Mpa, Poisson's ratio=0.48).
The pores have zero to negative stiffness.

For geometry modeling of the pore, the following variables or conditions are observed:
Each pore is modeled separately. Each pore cross section is a square with a variable side dimension in this example it is 433 μm.
The distance between the center of each pore can vary, in this example it is 722 μm.
The pore depth is taken from review of micropores OCT.
The thickness of the sclera is the average thickness measured in the same reference.

### Micropore Tool

In a non-limiting example, the micropore tool can have 3 different modes (although more is possible). The three modes are:
Pre-Planning Mode: Pretreatment step—calculate the PVF, accept as input. Perform an image recognition on the operative field for any obstructions that will obscure pore placement (blood vessels, foreign matter, etc.).
Treatment Mode: Peri treatment step—In real time, PVF and EMC is updated and displayed to the user as a color map.
Post Treatment Mode: Posttreatment step—Micropore Tool (which has the EMC calculator embedded), calculates relevant quantities and generates a report on the present pore matrix, each of the 4 pore matrices individually, and the 4 pore matrices as a system. MicroPore matrices can be independent, contiguous and arranged in a plurality of patterns. Micropores can also be reduced in size to Nanopores with the same plurailrt of configurations, shapes, sizes, and densities.

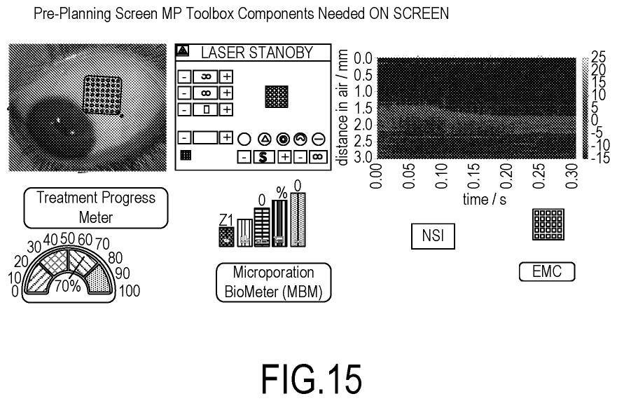

FIG. 15 illustrates a screenshot of the pre-planning mode, which includes, but not limited to:
Snapshot of an Individual's anatomical feature (e.g., eyeball).
Red Outline of the Matrix.
Has an Anatomy Avoidance Button.
Notes Button.
Edit/Markup/Fonts/Arrows/Drawing Toolbar.
EMC tell you the biomechanical property of the tissue.
PVFI tells you about the tissue removed, nothing about location.
Bulk Density Reading (BDR) tells you the regional stiffness.
Micropore Registration Tool (MIRT) tells you about the location of the pore, not the pattern.
Matrix Registration Tool (MART) tells you about the pattern.

The GUI can include one or more of the following tools:
Micropore Tool: A 3D Touchable Manipulatable GUI Simulation Tool that contains that accepts user inputs that define the parameters of the Ligament Efficiency Tool (LET) ASME. A tool that produces an intermediate result in the simulation solution.
Ligament Efficiency Tool (LET) (ASME): The physics package behind the micropore tool.
Registration Tool: (MPR)—(MAR) Location tracker which communicates & registers the location and orientation of the micropore & the Matrix.
Elastic Modulus Calculator: A processor of the following biomechanical and physics data inputs from the BioMeter.
Applications would be for 3 different uses. Pre-op planning, perioperative evaluation and validation, and post-op validation. A real-time GUI solver.
Microporation BioMeter (MBM): Individualized Biomechanical/Physics inputs to the EMC & 3D-ID VESA. The following inputs can be entered and/or measured:
   Components
   Age Related MpA
   Depth
   PVF
   Bulk Density (aka Regional Stiffness)
   Density
   Pore Size
   Pore Shape (Square) [Coding Language: Diameter]
   Pore Pattern
   Pore Array

The GUI of the micropore tool can also display one or more of the following features/values:
Negative Stiffness Index (NSI): Progress Bar indicating the Negative Stiffness Quotient of the biomechanical property/physics resultant change of scleral tissue during and after the LSM (Laser Scleral Microporation). For more detail on the LSM process, see more description below and FIGS. 65-70.
   Measured by Quadrant progression Q1-Q4 until total NSQ is met for the whole eye.
   Inputs are from the BioMeter+EMC
Treatment Progress Meter:
   Progressive completion meter regarding treatment progress this is also connect to a time clock for countdown of each quadrant backwards countdown clock is synchronized.
   Needs to be done by quadrant with a countdown incorporated.
Treatment Countdown Clock.
OCT Tool: Integration of AI and OCT Auto-Segmentation:
   Depth Sensing Tool to distinguish tissue layers.
   3D image processing and image presentation.
   3D depth analysis.
Custom Micropore Tool:
   A tray of micropore patterns to customize a treatment platform either by selection or by AI recommendation.
COP (Central Optical Power) Calculator:
   Uses a standard formula with a proprietary method to utilize Ray Tracing to calculate the COP of the crystalline lens.
   Calculates the free body forces of the lens.
   COP formulas and charts, see FIGS. 54-57.
Depth Sensor Tool: Innominds.

In some embodiments, the micropore tool is configured to determine material properties of tissue between pores. Once pore, depth, spacing, and diameter are determined, the entire structure of the eye can be simulated with the effect of the Micropore procedure. By determining the accommodation response, velocity can be obtained. Additionally, with the new micropore matrix in place, the new post treatment intraocular dynamic pressure can be obtained. This pressure can be compared against the pre-operative dynamic pressure.

The micropore tool is configured to determine the following:
Coefficient of Ocular Rigidity (COR): A mathematical formula predicting a constant.
Pore Volume Fraction Indicator (PVFI): A Mathematical formula predicting a pore bulk density and fractional tissue removed to % tissue remaining.
A GUI evaluation & validation tool that measures the PVF across the treatment area:
   PVF is relates toto scleral thickness by zone, scleral material properties, micropore shape, micropore density, and micropore size.
   Tells you about the tissue removed, but nothing about location.
Elastic Modulus Calculator (EMC): Relates to the Viscoeslastic and elastic properties of the tissue. Tells you the output of the Physics Meter in terms of Negative Stiffness changer & is a 3D simulation of the BioMeter.
The value of the physics Meter is the NSI. NSI relates to the negative poisson equation within the tissue.
The NSI is the trigger for recommendations.

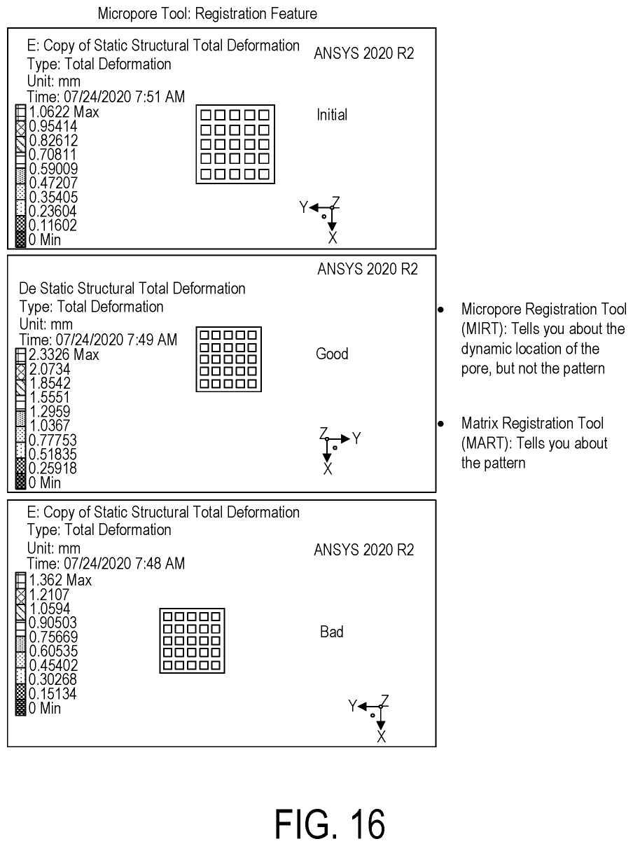

The micropore tool (MPTool) can also include a registration feature, as shown in FIG. 16. The micropore registration tool (MIRT) is configured to determine the dynamic location of the pore, but not the pattern. The matrix registration tool (MART) tells you about the pattern of the micropore. The MART and MIRT give continuous biofeedback to the data stack regarding the center of gravity of the eye ecosystem as it relates to NSI and biomechanical strain in the hoop stress coordinate map. The Hoop Stress Coordination Map (HSCM) gives locality, density, and registration information to the data base with continuous biofeedback to the data stack regarding these informations and other assigned information.

The micropore tool can also display one or more raw OCT images simultaneously such as, but not limited to, depth sensing images, y slice, z slice, and x slice, in 1D, 2D, 3D, and 4D viewer capacities for example. The MPTool also has zooming capabilities for macro and micro viewing and evaluation. The GUI of micropore tool is also configured to overlay key tissues on the images. Key tissues that can be overlaid include, but not limited to, cornea, sclera, conjunctiva, ciliary muscle fiber, and tenon's capsule. This enables easy diagnosis.

The micropore tool has 5 main modes of operation:
Pre-Treatment Planning (Snapshot).
Treatment (Dynamic Real-time).
Post Treatment Immediate (Simulation).
Post Treatment Post Processing.
Evaluation of the Pore Matrix & Reporting (Simulation).

During all Modes, there is a need for a simulation of the matrix in terms of (PVF/BD/EM/COR). This will help the medical professional to:
Plan the matrix beforehand.
Evaluate each of the 4 pore matrices as the laser procedure is being performed. This will allow for re-lasing of areas that do not meet guidelines & inform user of suggested correction.
See Real time dynamic changes in ocular tissue as a result of the LSM therapy.
Report the responsible parties the results of the intervention or lasing procedure to determine procedure success.

In the Peri Treatment step, this needs to be done in real time, which makes an FEA simulation unsuitable. As such, the preoperative VESA sim is done with various 3D-ID™—DOE Approach (Design of Experiments). Further iterations of the VESA FEA are available through the cloud applications.

The micropore tool can include an EMC calculator, which is based upon a first-principals based calculation of stress (ASME Ligament Efficiency) that enable stress calculations to be solved in real time.

### Pre-Scan Visit Process

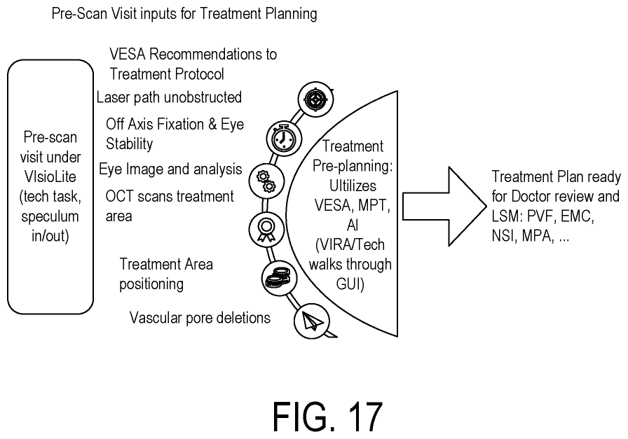

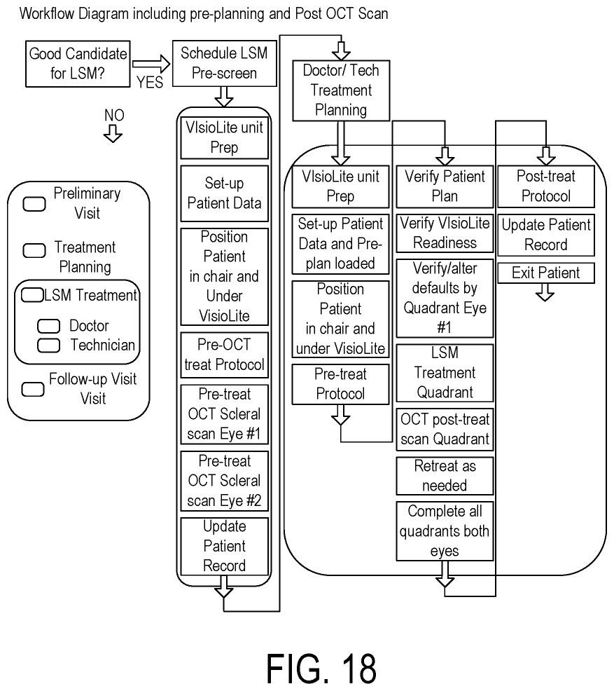

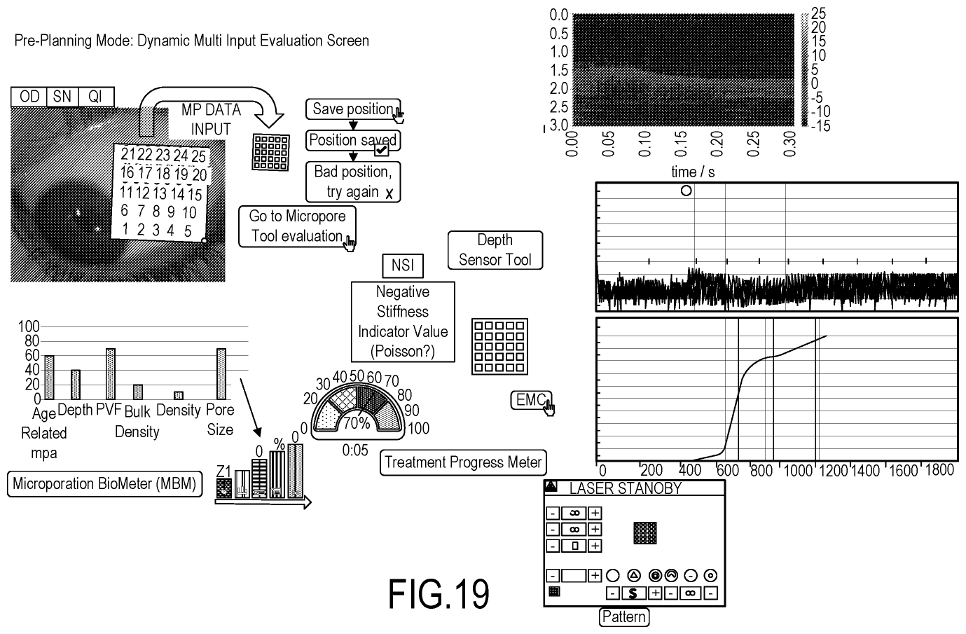

The pre-scan visit process can include steps and processes as shown on FIG. 17, and both the pre-planning and post OCT scan workflow is shown in FIG. 18. An application having a plurality of GUIs and features configured to facilitate the individual intaking process is shown in FIG. 19.

From the screen shown in FIG. 19, the user can:
Perform registration of position with eye tracker.
Calibrate laser.
Step 1. Set up Position.
Step 2. Run Simulation for Estimated NSI (negative stiffness indicator value) post treatment Matrix Q1.
Step 3. Confirmation Position.
Step 4: Go to the Edit Screen or Treatment Screen or various sub screens.

With respect to the micropore array, the user can:
Select/Deselect/Add Pores.
Run simulation.
Perform live PVF calculation.
Perform viewing and evaluation.
Perform live LET, NSI, and EMC calculations.
View live elasticity pore placement assumptions.
Receive live AI recommendations based on BioMeter/EMC/NSI.

The screen of FIG. 19 can also include a physics dynamic progress meter, which measure:
PVF.
Bulk Density.
Negative Stiffness Index (NSI).
Elastic Modulus.
Depth.

The micropore tool can include a treatment-mode screen, which can include a real-time OCT signal chart.

The micropore tool can perform simulation and include sub-screens. The VESA simulation can be used to predict the outcomes and set the GO NO GO parameters. VESA simulation within the MPT will run the simulation under these scenarios:

Pre Planning—If Edits are performed VESA will control the stop edit/VESA will allow to progress to next quadrant with subtracted pores so long as the Embedded EMC code will reach the desired NSI in order to create a successful individual Matrix.
Progressive matrices will be running in the background and allow the surgeon to bypass Go to MPT if the series of matrices still meet the NSI value whole globe requirement predicted by VESA when the treatment is completed.
Force the Retreat when the OPF ratio is not met in each quadrant using the MRT to return to the registered Pore.
Force an ADD ON Pore array when the threshold is met if edits of the matrix require micropore replacement array.
Act as a medical enforcement control center to prevent unwanted treatment parameters or interventions.
Act as a notification agent for biomechanical, therapeutic, or surgical intervention parameters and conditions.
Act as a stop system mechanism or abort mechanism.

The micropore tool can include a post treatment screen (FIGS. 53A-53B). For post treatment, NSI needs to have thresholds 1) GO/ 2) Consider Retreat 3) NO GO should change color accordingly.

### Hyper-Elastic Image Registration

The hyperelastic modeling in Ansys enables tracking of critical points of the numerical model while the accommodation is simulated. These critical points can also be tracked on life imaging, therefore allowing confirmation of the accuracy of our model with multiple data points.

In hyperelastic modeling, reverse imaging of dynamic biological movements can be translated into a 3D FEM. Additionally, determination of stresses within the deforming body can be computed.

### Eye Tracking Augmented Reality

VESA can include a system for eye tracking with augmented reality.

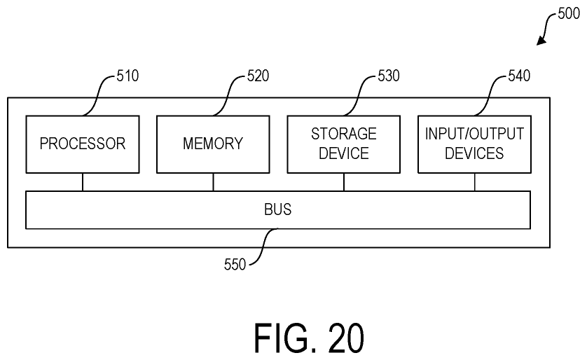

FIG. 20 depicts a block diagram illustrating an example of a computing system 500 consistent with implementations of the current subject matter. The computing system 500 may implement VESA, 3D-ID AI, MP Tool, and/or any components therein. The system can include or be coupled to an imaging system, a biothermal system, an interactive user interface, and/or an inputs program to receive and manipulate physical, biometric, biomechanical, material and mechanical information and data.

As shown in FIG. 20, the computing system 500 can include a processor 510, a memory 520, a storage device 530, and input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, VESA, 3D-ID AI, and/or MP Tool. In some implementations of the current subject matter, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a digital cloud, a hard disk device, an optical disk device, or a tape device, or other suitable persistent storage means. The input/output device 540 provides input/output operations for the computing system 500. In some implementations of the current subject matter, the input/output device 540 includes a keyboard and/or pointing device. In various implementations, the input/output device 540 includes a display unit for displaying graphical user interfaces.

According to some implementations of the current subject matter, the input/output device 540 can provide input/output operations for a network device. For example, the input/output device 540 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 500 can be used to execute various interactive computer software applications that can be used for organization, analysis and/or storage of data in various (e.g., tabular) format (e.g., Microsoft Excel®, and/or any other type of software). Alternatively, the computing system 500 can be used to execute any type of software application. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug-ins, or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.). The user interface can be integrated with other devices or virtual ecosystems.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, joy sticks, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system for recreation, manipulation and intervention of an individual's eye in virtual reality comprising: a 3D modeling component comprising of:
at least one data processor;
an imaging system;
a biothermal system;
an interactive user interface;
an inputs program to receive and manipulate physical, biometric, biomechanical, material and mechanical information and data; and
at least one memory storing instructions, which when executed by the at least one data processor, result in operations comprising:
generating a first specific simulation in which the Bruch's Membrane Choroid apparatus is activated to determine the first contribution of the energy required for deformation of the lens;
generating a second individual-specific simulation in which a origin section of the ciliary muscle fiber section of an eye of the individual is activated to determine a first contribution of the first ciliary muscle fiber section to a deformation of a lens of the eye;
generating a second individual-specific simulation in which a second ciliary muscle fiber section of the eye of the individual is activated to determine a second contribution of the second ciliary muscle fiber section to the deformation of the lens of the eye;
generating a third individual-specific simulation in which a third ciliary muscle fiber section of the eye of the individual is activated to determine a third contribution of the second ciliary muscle fiber section to the deformation of the lens of the eye; and
determining, based at least on the first Bruch's Choroid apparatus, the first contribution of the first ciliary muscle fiber section, the second contribution of the second ciliary muscle fiber section, the third contribution of the third ciliary muscle fiber section, one or more parameters of a treatment for the individual.

2. The system of claim 1, wherein all simulations are capable of being performed, in sequence, in isolation and/or in parallel with other parameter manipulations.

3. The system of claim 1, wherein it results in comprising of:
generating a fluid structure interaction of the aqueous flow of the eye in which three outflow pathways are activated.

4. The system of claim 1, wherein the system includes one or more parameters of the Bruch's Choroid Apparatus, the first ciliary muscle fiber section, the second ciliary muscle fiber section, and the third ciliary muscle fiber section of the eye of the individual.

5. The system of claim 4, wherein the one or more micropores are generated into the scleral tissue of the eye over the Bruch's Choroid apparatus, the first ciliary muscle fiber section and/or the second ciliary muscle fiber section and/or third ciliary muscle fiber section of the eye of the individual by laser.

6. The system of claim 4, wherein the one or more parameters of the treatment includes a quantity of matrices including the one or more pores, a placement of the matrices, an overall shape of each matrix, an overall dimension of each matrix, a quantity of pores in each matrix, a distribution of pores in each matrix, and/or a depth of pores in each matrix.

7. The system of claim 1, wherein the one or more treatment parameters for the individual are determined by applying a machine learning model.

8. The system of claim 7, wherein the machine learning model comprises a regression model and algorithm.

9. The system of claim 7, wherein the machine learning model comprises an age progression model and algorithm.

10. The system of claim 7, wherein the machine learning model is trained based on various combinations of treatment parameters and treatment outcomes.

11. The system of claim 9, wherein the machine learning model is further trained based individual demographics data associated with the various combinations of treatment parameters and treatment outcomes.

12. The system of claim 7, wherein the operations further comprise:
generating a user interface displaying one or more parameters of the treatment for the individual.

13. The system of claim 11, wherein the operations further comprise:
receiving, via the user interface, at least one user input associated with the one or more parameters of the treatment for the individual; and
updating, based at least on the user input, the machine learning model.

13. The system in claim 1, wherein the operations further comprise systems and method for provided for evaluating a biomechanical property of tissue.

14. The system in claim 1, wherein an evaluation toolbox component is configured to calculate at least one parameter associated with the various possible outcomes of a given treatment intervention in an individual's eye from the extracted plurality of features. A system output is configured to provide the calculated at least one parameter to one of a treatment system and a user. One or more optical, biometric, biomechanical, physical, and geometrical parameters are incorporated into the calculation prediction.

15. A system and method as in claim 1 wherein, the calculator toolbox of VESA is capable of reconciling equations that produce the most optimal solution(s) therapeutic or surgical intervention to the user.

16. A system for evaluating an eye of a patient, comprising:
a modeling component configured to determine a representation of the whole eye from a three-dimensional structural image of the whole eye and at least one biomechanical property of the eye;
a feature extractor configured to extract a plurality of features from the model of the whole eye;
a user interface configured to accept input from a clinician defining an objective function as a function of at least one parameter for the eye after the therapeutic procedure;
an condition or disease evaluation component configured to calculate at least one parameter associated with the risk of the disease in the eye from the extracted plurality of features and the objective function, the calculated at least one parameter including a variable in a therapeutic procedure representing a surgical parameter that can be varied by a clinician in the therapeutic procedure; and
a system output configured to provide the calculated at least one parameter to one of a treatment system and a user.

17. The system of claim 16, the modeling component being configured to determine a three-dimensional finite element model of the whole eye from the three-dimensional structural image and at least one biomechanical property of the eye.

18. The system of claim 17, wherein the modeling component is configured to provide a three-dimensional finite element model representing the whole eye after the therapeutic procedure, the system further comprising a user interface configured to accept input from a clinician defining at least a type and location of the therapeutic procedure.

19. The system of claim 18, wherein the feature extractor is configured to extract the at least one feature of the plurality of features from the three-dimensional finite element model representing the whole eye after the therapeutic procedure, the ectasia evaluation component being configured to calculate a parameter representing an expected risk of ectasia to the patient given a therapeutic procedure having the associated type and location.

20. The system of claim 19, the ectasia evaluation component being configured to perform a sensitivity analysis on at least one feature, such that a magnitude of an impact of the value of the at least one feature on the at least one parameter can be determined.

21. The system of claim 19, wherein the extracted at least one feature represents one of a geometric or biomechanical characteristic of the eye.

22. The system of claim 17, wherein the modeling component is configured to provide a three-dimensional finite element model representing the whole eye with a load applied to the eye, the system further comprising a user interface configured to accept input from a clinician defining at least a magnitude and location of the load.

23. The system of claim 16, the modeling component being configured to determine a finite element model of the whole eye from the three-dimensional structural image and at least one biomechanical property of the eye.

24. The system of claim 16, wherein the objective function is a function of at least one of a strain value of the eye and a stress value of the eye.

25. The system of claim 16, wherein the objective function is a function of at least one measure of refractive outcome.

26. The system of claim 16, wherein the modeling component determines the representation of the whole eye as a virtual model comprising a set of parameters extracted from evaluating a statistical model according to the three-dimensional structural image and at least one biomechanical property of the eye.

27. A system for recreation, manipulation and intervention of an individual's eye in virtual reality comprising: a 3D modeling component comprising of:
at least one data processor;
an imaging system, a biothermal system, an interactive user interface; an inputs program to receive and manipulate physical, biometric, biomechanical, material and mechanical information and data; and
at least one memory storing instructions, which when executed by the at least one data processor, result in operations comprising:
generating a first specific simulation in which the Bruch's Membrane Choroid Complex apparatus is activated to determine the first contribution of the energy required for deformation of the lens;
generating a second individual-specific simulation in which a origin section of the zonule section of an eye of the individual is activated to determine a first contribution of the first zonule section to a deformation of a lens of the eye;
generating a second individual-specific simulation in which a second zonule section of the eye of the individual is activated to determine a second contribution of the second zonule section to the deformation of the lens of the eye;
generating a third individual-specific simulation in which a third zonule section of the eye of the individual is activated to determine a third contribution of the second zonule section to the deformation of the lens of the eye; and
determining, based at least on the first Bruch's Choroid apparatus, the first contribution of the first zonule section section, the second contribution of the second zonule section section, the third contribution of the third zonule section section, one or more parameters of a treatment for the individual.
